(19) United States
(12) Patent Application Publication
(10) Pub. No.: US 2009/0036954 A1
(43) Pub. Date: Feb. 5, 2009

(54) INSTRUMENT FOR TREATING SCALP AFFECTIONS

(75) Inventors: Cesare Ragazzi, Zola Predosa (IT); Jacopo Talluri, Firenze (IT)

Correspondence Address: WEINER & BURT, P.C., 635 N US-23, POB 186, HARRISVILLE, MI, 48740, US

(73) Assignee: CESARE RAGAZZI COMPANY S.p.A., Bologna (IT)

(21) Appl. No.: 12/280,746

(22) PCT Filed: Feb. 20, 2007
(86) PCT No.: PCT/EP07/51587

§ 371 Date: Sep. 26, 2008

(30) Foreign Application Priority Data
Feb. 27, 2006 (IT) FI2006A000053

(51) Int. Cl. A61N 5/06

(52) U.S. Cl. 607/88

(57) ABSTRACT

An instrument (1) for treating scalp affections, comprising a shell (5) from a portion of which a plurality of protrusions (2) protrude, another portion being constituted by a handle (7). The instrument (1) is substantially shaped like a sort of brush. The protrusions (2) comprise, at their terminal end, respective electromagnetic radiation emitter.

### TECHNICAL FIELD

The present invention relates to an instrument for treating scalp affections.

### BACKGROUND ART

It is known to use skin treatment devices provided with light sources of various kinds: subjecting the skin to cycles of successive lighting, with radiation having a certain wavelength and a predefined intensity, entails stimulation of the cells with consequent increases in skin elasticity and greater blood flow.

It is also known to use localized massages (performed manually or by means of appropriate devices) or preparations of various kinds (which often have a revulsive effect) for stimulating the scalp in order to improve the appearance and/or “health” of hair.

### DISCLOSURE OF THE INVENTION

The aim of the present invention is to provide an instrument for treating scalp affections which is adapted for stimulation of the scalp with light radiation.

Within this aim, an object of the present invention is to provide an instrument for treating scalp affections which has a low cost, is relatively simple to provide in practice and safe in application.

This aim and this and other objects which will become better apparent hereinafter are achieved by the present instrument for treating scalp affections, which comprises a shell from a portion of which a plurality of protrusions protrude, another portion being constituted by a handle, said instrument being substantially shaped like a sort of brush, characterized in that said protrusions comprise at their terminal end respective electromagnetic radiation emitters.

### BRIEF DESCRIPTION OF THE DRAWINGS

Further characteristics and advantages of the invention will become better apparent from the following detailed description of a preferred but not exclusive embodiment of an instrument for treating scalp affections, illustrated by way of non-limiting example in the accompanying drawings, wherein:

FIG. 1 is a top view of an instrument for treating scalp affections according to the invention;

FIG. 2 is a top view of a portion of an instrument for treating scalp affections according to the invention according to an alternative embodiment;

FIG. 3 is a top view of a portion of an instrument for treating scalp affections according to the invention according to an alternative embodiment;

FIG. 4 is a side view of an instrument for treating scalp affections according to the invention.

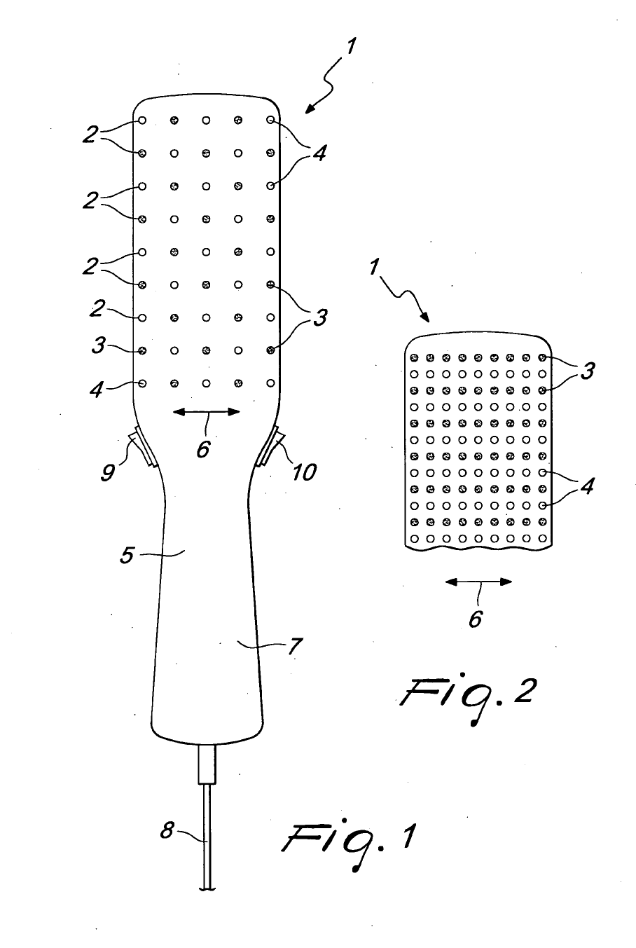

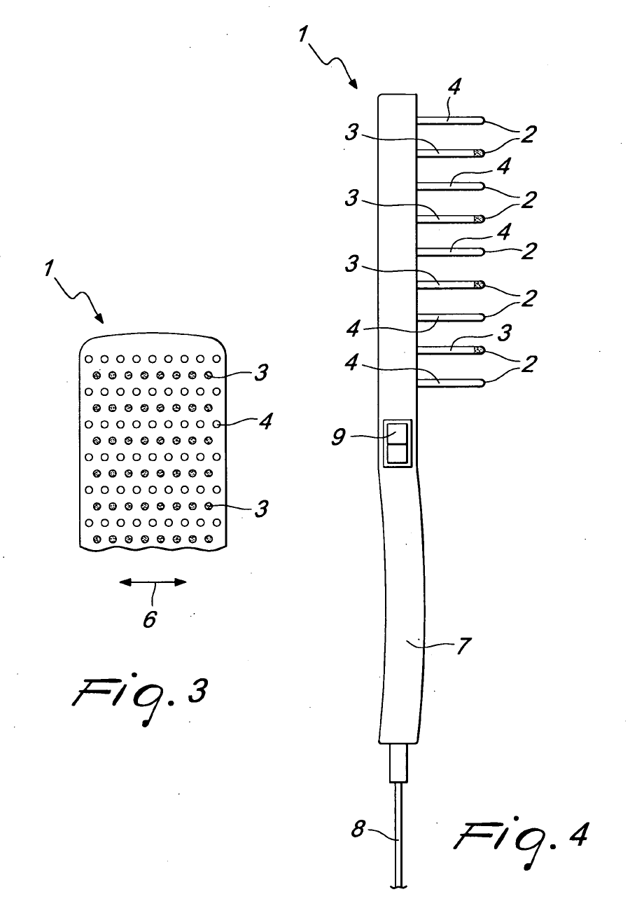

### WAYS OF CARRYING OUT THE INVENTION

With reference to the figures, the reference numeral 1 generally designates an instrument for treating scalp affections.

The instrument 1 is of the type of a brush, a comb or other equivalent element, which comprises protrusions 2 which emit light stimuli: according to a particular constructive embodiment, some protrusions 3 emit stimulating radiation in the near infrared (NIR) range and protrusions 4 emit stimulating radiation in the ultraviolet (UV) range.

The stimulations of each of the two types are preferably emitted in a pulsed and asynchronous manner, although it is possible to provide the possibility to adjust the period and synchronization of the pulses completely, according to requirements.

In order to optimize the result on the scalp, the light stimulations can have a power of more than one milliwatt per spot of light.

Advantageously, according to a particular embodiment of unquestionable practical interest, the stimulations of one of the two types can be emitted in a staggered manner with respect to the stimulations of the other type, and preferably the pulsed stimulations of one type are emitted during the intervals between the pulsed stimulations of the other type.

The pulsed stimulations of one type can be prolonged for different times with respect to the duration of the subsequent pulsed stimulations of the other type.

The distribution of the protrusions 3 with respect to the protrusions 4 may be any according to constructive requirements and depending on the type of final application for which the instrument 1 is intended.

According to a particular embodiment of unquestionable interest in application, the instrument 1 can have protrusions 3 and 4 of the two types distributed in alternating rows. As an alternative, the protrusions 3 and 4 of the two types can be distributed along each of a plurality of rows of protrusions 2, with an optional staggering between the protrusions 3 and 4 of the two types provided in contiguous rows.

Light stimulation of the hair bulb with coherent light emitted in the near infrared (NIR) has been shown to be able to induce positive results on hair growth; the effects of this treatment can be amplified further by adapted manipulation of the signal according to appropriate cyclic intermittences.

It is known in fact that in nature all biological effects can be stimulated conveniently by means of specific applications of energy charges, provided that said energy charges are not applied continuously.

On the other hand, it is known that problems affecting the nearby skin, such as psoriasis, dandruff or other skin affections, are often present in association with the disorder of the hair and of the hair bulb. These affections can be treated with positive results by phototherapy with radiation in the ultraviolet (UV) range.

The protrusions 3 and 4 designed to emit light radiation each comprise at least one respective light emitting diode, known commercially as LED, whose spectrum of emission is in the near infrared (NIR) or in the ultraviolet (UV).

The emissions of the various LED sources, according to what has been cited above, are of the pulsed and asynchronous type, so as to act on the two different affections without mutual interference.

The instrument 1 is constituted by a shell 5 which can be gripped and from which the protrusions 2 (of the type 3 and of the type 4) protrude: the overall appearance of the shell 5 and its protrusions 2 is substantially the appearance of a brush (the protrusions 2 can be likened to the bristles of such brush).

With reference to the instrument 1 shown in the accompanying figures, a movement which is substantially performed along a direction indicated by the arrows 6 is assumed during use. The instrument 1 is held by the operator by means of the handle 7, which also can be likened substantially to the handle of a brush.

The instrument 1 comprises a power supply cable 8 and a control and management circuit (not shown in the figure) for the LEDs of the two types: said circuit is intended to adjust the synchronization and period (cycle) of the light pulse of the emissions.

The power supply cable 8 can be adapted for the direct supply of power of the device but can also be a cable. 8 for recharging an internal battery (not shown in the figure) designed to dispense the energy required for operation: in this second solution, maximum safety of the user against risks linked to electrical shocks is ensured.

The circuit can be arranged equally inside the shell 5 (accommodated within an appropriately provided internal seat thereof) or arranged within a respective external unit to which the cable 8 leads.

In either case, the circuit is of the type normally used for management and control of LED light sources and can be equipped positively with units for adjusting the duration of the period of the pulse and for adjusting the switch-on offset between the LEDs accommodated on the protrusions 3 with respect to the ones accommodated on the protrusions 4.

According to an embodiment of particular practical and application-related interest, the instrument 1 can also operate for individual treatment with near infrared (NIR) radiation or with ultraviolet (UV) radiation; for this reason, there are selectors 9 and 10 by means of which it is possible to activate or exclude one or the other of the protrusions (3 and/or 4). As an alternative, this selection can also be performed by acting directly on the control and management circuit by means of adapted control elements which are not shown in the figure.

During use, the instrument 1 is used like an ordinary brush, thus arranging the terminal portions of the protrusions 3 and 4 so that they face the scalp, with a movement along the direction indicated by the arrows 6 in the figure.

This type of application ensures intense stimulation of the scalp, with a consequent improvement of the health of the treated hair and skin.

The instrument 1 shown in the figure is substantially shaped like a brush: this embodiment is to be considered absolutely as an example, since the shape will be dictated by ergonomic requirements which are strictly linked to the type of treatment for which the instrument 1 is intended.

It has thus been shown that the invention achieves the intended aim and object.

The invention thus conceived is susceptible of numerous modifications and variations, all of which are within the scope of the appended claims.

All the details may further be replaced with other technically equivalent ones.

In the exemplary embodiments shown, individual characteristics, given in relation to specific examples, may actually be interchanged with other different characteristics that exist in other exemplary embodiments.

Moreover, it is noted that anything found to be already known during the patenting process is understood not to be claimed and to be the subject of a disclaimer.

In practice, the materials used, as well as the shapes and dimensions, may be any according to requirements without thereby abandoning the scope of the protection of the appended claims.

The disclosures in Italian Patent Application No. F12006A000053 from which this application claims priority are incorporated herein by reference.

1. An instrument for treating scalp affections, comprising;
a shell (5) from a portion of which a plurality of protrusions (2) protrude, and another portion being constituted by a handle (7);
said instrument (1) being substantially shaped like a sort of brush;
said protrusions (2) comprising at their terminal end respective electromagnetic radiation emitters; and
said radiation emitters emit radiation in a pulsed manner.

2. The instrument according to claim 1, characterized in that said protrusions (2) comprise a plurality of protrusions (3) provided with emitters of radiation in the near infrared range and protrusion (4) provided with radiation emitters in the ultraviolet (UV) range.

3. The instrument according to claim 1, characterized in that said radiation emitters are light-emitting diodes, ordinarily known as LED diodes.

4. The instrument according to claim 1, including:
a circuit for the control and management of said radiation emitters which is associated with an electric power source and is provided with respective control means (9, 10).

5. The instrument according to claim 4, characterized in that said control and management circuit comprises appropriate timers and appropriate signal generators for the controlled intermittent supply of power to the emitters.

6. The instrument according to claim 2, characterized in that the radiation emissions of one group of emitters are of the pulsed intermittent type, said radiation emission being active during the interval between two successive emissions of the other group of emitters.

7. The instrument according to claim 2, characterized in that the duration and intensity of radiation emission of the emitters of one group, LED diodes with an emission spectrum in the near infrared, are substantially independent of the duration and radiation emission intensity of the emitters of another group, LED diodes with an ultraviolet emission spectrum.

8. The instrument according to claim 1, characterized in that the protrusions are distributed in alternating rows.

9. The instrument according to claim 1, characterized in that the protrusions are distributed uniformly along a same surface of said shell (5), with an offset between the protrusions provided in contiguous rows.

10. The instrument according to claim 6, characterized in that the duration and intensity of radiation emission of the emitters of one group, LED diodes with an emission spectrum in the near infrared, are substantially independent of the duration and radiation emission intensity of the emitters of another group, LED diodes with an ultraviolet emission spectrum.

11. The instrument according to claim 2, characterized in that the protrusions are distributed in alternating rows.

12. The instrument according to claim 3, characterized in that the protrusions are distributed in alternating rows.

13. The instrument according to claim 2, characterized in that the protrusions are distributed uniformly along a same surface of said shell (5), with an offset between the protrusions provided in contiguous rows.

14. The instrument according to claim 3, characterized in that the protrusions are distributed uniformly along a same surface of said shell (5), with an offset between the protrusions provided in contiguous rows.

15. The instrument according to claim 4, characterized in that the protrusions are distributed uniformly along a same surface of said shell (5), with an offset between the protrusions provided in contiguous rows.

16. The instrument according to claim 5, characterized in that the protrusions are distributed uniformly along a same surface of said shell (5), with an offset between the protrusions provided in contiguous rows.

17. The instrument according to claim 6, characterized in that the protrusions are distributed uniformly along a same surface of said shell (5), with an offset between the protrusions provided in contiguous rows.

18. The instrument according to claim 7, characterized in that the protrusions are distributed uniformly along a same surface of said shell (5), with an offset between the protrusions provided in contiguous rows.

19. The instrument according to claim 8, characterized in that the protrusions are distributed uniformly along a same surface of said shell (5), with an offset between the protrusions provided in contiguous rows.

20. The instrument according to claim 7, characterized in that the protrusions are distributed in alternating rows.
